(19) United States
(12) Patent Application Publication
(10) Pub. No.: US 2023/0140282 A1
(43) Pub. Date: May 4, 2023

(54) BLOOD FLOW RESTRICTION DEVICE

(71) Applicants: Chett B. Paulsen, South Jordan, UT (US); Robert Rossetti, Portsmouth, NH (US)

(72) Inventors: Chett B. Paulsen, South Jordan, UT (US); Robert Rossetti, Portsmouth, NH (US)

(21) Appl. No.: 17/378,475

(22) PCT Filed: Jan. 17, 2020
(86) PCT No.: PCT/US2020/014102

§ 371 Date: Jul. 16, 2021

Related U.S. Application Data
(60) Provisional application No. 62/794,114, filed on Jan. 18, 2019.

(51) Int. Cl. A61B 17/132

(52) U.S. Cl. CPC A61B 17/1327; A61B 2017/00022

(57) ABSTRACT

A blood flow restriction (BFR) device includes a cuff and a tightening system coupled to the cuff. The tightening system is configured to tighten and loosen the cuff. The BFR device may include a silicon-based cushion disposed on an inner surface of the cuff. The cuff may include an outer structure and an interior structure. The tightening system may include a pull wire disposed between the outer structure and the interior structure. The tightening system may further include a spool coupled to the pull wire and a bezel disposed on the spool. The bezel may be configured to rotate to tighten the BFR device. The BFR device may include a limb occlusion pressure (LOP) sensor disposed on an inner surface of the cuff.

### RELATED APPLICATION

This application claims benefit under 35 U.S.C. § 371 of International Patent Application No. PCT/US2020/014102, filed Jan. 17, 2020, which claims the benefit of U.S. Provisional Application No. 62/794,114, filed Jan. 18, 2019, which are incorporated by reference in their entirety.

### TECHNICAL FIELD

The present disclosure relates to restriction devices, and, more particularly to blood flow restriction devices (also referred to as ischemia devices).

### BACKGROUND

In blood flow restriction (BFR) training (e.g., occlusion training), a device (e.g., cuff, band) may be disposed around a portion of the body (e.g., a limb) during exercise to maintain arterial inflow to a muscle while preventing venous return. Due to the restricted blood flow, lighter loads in BFR training can have similar results compared to higher loads during regular high intensity interval training (HIIT) exercise.

### DESCRIPTION OF THE DRAWINGS

The present disclosure is illustrated by way of example, and not by way of limitation in the figures of the accompanying drawings.

FIGS. 1A-B illustrate blood flow restriction (BFR) devices, according to certain embodiments.

FIG. 1C illustrates a tightening system for a BFR device, according to certain embodiments.

FIGS. 2A-2C illustrate closure systems for BFR devices, according to certain embodiments.

FIG. 3 illustrates a BFR device including a sensor and a microcontroller, according to certain embodiments.

FIGS. 4A-4E illustrate silicon-based cushions for BFR devices, according to certain embodiments.

FIG. 4F illustrates a mold for a pad for a BFR device, according to certain embodiments.

FIGS. 5A-5H illustrate tightening systems of BFR devices, according to certain embodiments.

FIGS. 6A-6C illustrate closure systems for BFR devices, according to certain embodiments.

FIG. 7 illustrates a BFR device, according to certain embodiments.

FIGS. 8A-8B illustrate BFR devices, according to certain embodiments.

FIG. 8C illustrates a closure system for a BFR device, according to certain embodiments.

FIG. 9 illustrates the percent of occlusion in a chart, according to certain embodiments.

FIGS. 10A-10D illustrate BFR devices, according to certain embodiments.

FIG. 11A illustrates a strap for a BFR device, according to certain embodiments.

FIG. 11B illustrates a measuring tape for a strap of a BFR device, according to certain embodiments.

FIGS. 12A-12D illustrate BFR devices, according to certain embodiments.

FIG. 13 is a diagrammatic representation of a machine in the example form of a computer system including a set of instructions executable by a computer system, according to any one or more of the methodologies discussed herein.

### DETAILED DESCRIPTION

Described herein are systems and devices directed to blood flow restriction (BFR). For BFR training (BFRT) (e.g., occlusion training), a device may be attached to a part of the body (e.g., upper thigh, upper arm, leg, arm, wrist, and/or the like). The device may provide BFR that partially occludes arterial blood flow and substantially or completely occludes venous blood flow. By providing BFR, lower loads (e.g., lighter weight, lower intensity, less repetition, and the like) may have similar or better results compared higher loads without BFR (e.g., in uses of rehabilitation after surgery/injury, general fitness, strength and conditioning, and with “at-risk” populations).

A conventional way of providing BFR is by using pneumatic surgical cuffs. Surgical cuffs may be painful, expensive, and inconvenient to use. To use a surgical cuff, a user may require active monitoring by a healthcare professional and may be tethered to an electronic box. Used without supervision or proper education, a surgical cuff may fully occlude arterial pressure causing deep vein thrombosis (DVT); or, tightened too much, may cause nerve damage, rhabdomyolysis, user pain, or reduced nerve conduction velocity.

Another conventional way of providing BFR is by using a device with a chambered air bladder. Use of a device with a chambered air bladder is inconvenient to use and may require that the user actively test limb occlusion pressure (LOP) or reference an application (app) to obtain proper pressures. After use, the air bladder is to be fully bled to measure consistent pressure when inflating as partially deflated cuffs can provide false readings and a user's variable systolic pressure can alter the effective range.

Another conventional way of providing BFR is by manually tightening a non-pneumatic band (tourniquet), sash, or surgical tubing around a portion of the body. Use and design of bands, sashes, and surgical tubing may be dangerous because they may cause DVT, nerve damage, pain, rhabdomyolysis, and more, in addition to not providing the intended effect other than by chance.

The systems, devices, and methods described herein provide a blood flow restriction (BFR) device. The BFR device, as described herein, may include a cuff, a tightening system for tightening and loosening of the cuff, and a pad (e.g., silicon-based cushion, a platinum-based silicon material, etc.) disposed on an inner surface of the cuff. The cuff may include an outer structure (e.g., fabric sleeve) and an interior structure (e.g., a flexible plastic structure). The tightening system may include a pull wire disposed between the outer structure and the inner structure. The tightening system may further include a spool coupled to a first distal end of the pull wire, a rivet connected to a second distal end of the pull wire, and a bezel connected to the spool. The bezel may be configured to rotate to tighten the BFR device. An LOP sensor may be disposed on an inner surface of the cuff to provide an indication of one or more of an LOP reading, active pressure, or pulse.

The bezel may limit how much arterial blood flow may be occluded (e.g., a user may not be able to rotate the bezel to completely occlude arterial blood flow), thereby not requiring active monitoring by a health care professional, not requiring being tethered to an electronic box, and not being dangerous to the user. By using the silicon-based cushion instead of an air bladder, a user may remove and attach the BFR device without waiting to deflate and inflate and may avoid false readings of partially deflated cuffs. The inclusion of an LOP sensor may provide the LOP readings to a user without necessitating connection to an electrical box, without use of an app, and without stopping exercise to attach a sensor. The silicon-based cushion can be one or more of certified “skin safe,” infused with silver particles to reduce bacteria and smell, or be removed from the cuff for cleaning or replacement with a like cushion of different durometer.

The BFR device, as disclosed herein may be used for one or more of physical therapy, athletic training, occupational therapy, strength and conditioning, and the like. The BFR device may be used by elite professional athletes, Olympic athletes, college athletes, geriatric people, people undergoing physical therapy, and other users.

Use of the BFR device, compared to conventional exercise without BFR, may one or more of reduce time required to work out, reduce time for after-surgery rehabilitation, increase athletic performance, provide an alternative to high-intensity training for “at risk” populations like geriatric populations, increase muscle size, reduce atrophy in non-injured limbs, reduce chance of injury from high weight loads or repetitions, e.g., by using BFR +LI (low intensity) instead of HIT (high intensity training), and the like.

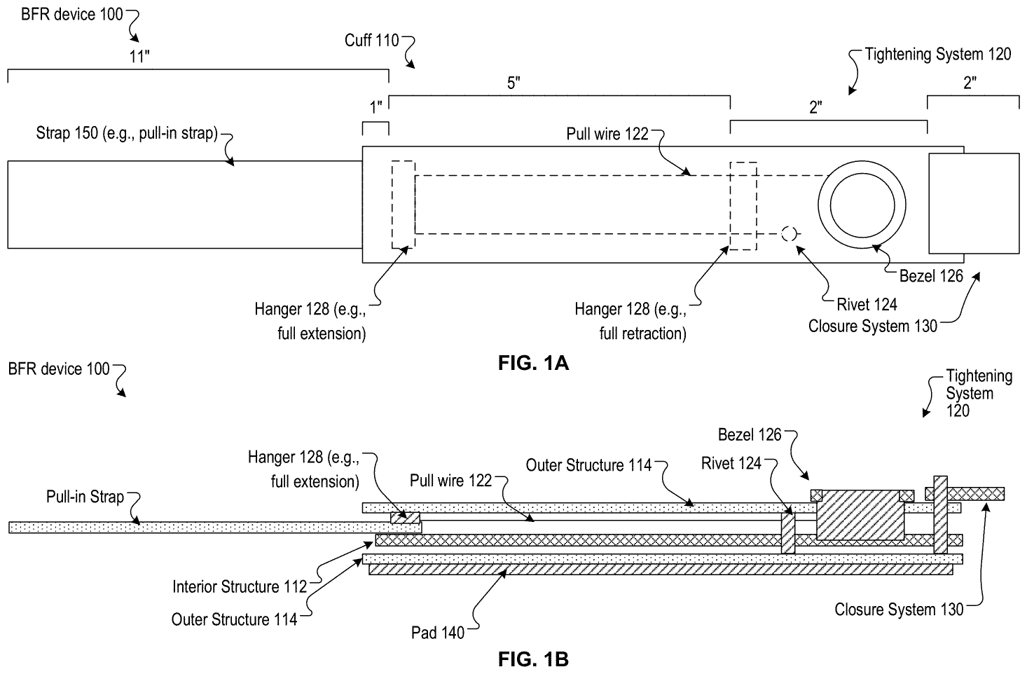

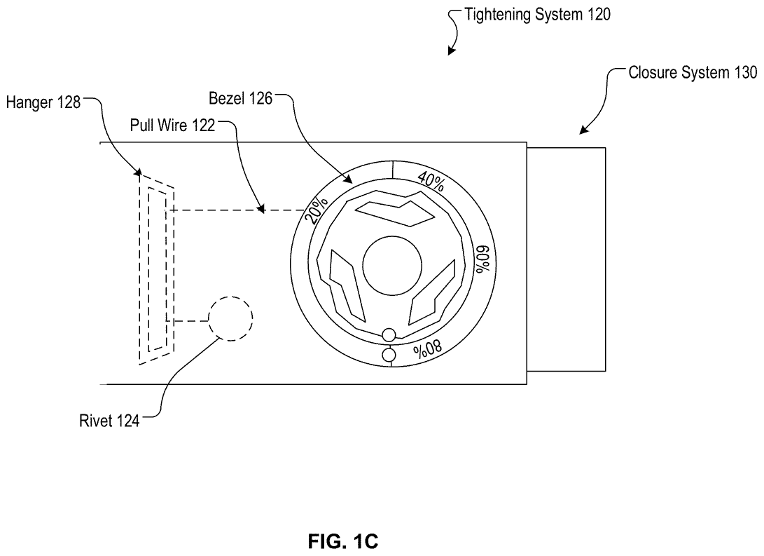

FIGS. 1A-B illustrate a BFR devices 100, according to certain embodiments. In some embodiments, FIGS. 1A-B illustrate the same BFR device 100 or similar BFR devices 100. FIG. 1A illustrates a top view of a BFR device 100, according to certain embodiments. FIG. 1B illustrates a cross-sectional side view of a BFR device 100, according to certain embodiments. The BFR device 100 may be configured to circumscribe a portion of a body. For example, a BFR device 100 may be configured to circumscribe one or more of a leg, an arm, a wrist, etc.

The BFR device 100 includes a cuff 110. The cuff 110 may include an outer structure 114 and an interior structure 112 (e.g., shield structure). The outer structure 114 be made of a fabric (e.g., woven fabric). The interior structure 112 may be made of a plastic, such as high density polyethylene (HDPE). The cuff 110 may be a band, strap, wrap, injection molded plastic, etc. The outer structure 114 may be an outer layer of the BFR device 100. In some embodiments, the cuff 110 has an outer structure 114 (e.g., fabric) and an interior structure 112 (e.g. shield structure, plastic structure, rigid structure, malleable plastic shield structure, etc.) disposed within the outer structure 114. For example, the outer structure 114 may be a fabric sleeve and the interior structure may be a flexible plastic structure (e.g., rectangular sheet of flexible plastic).

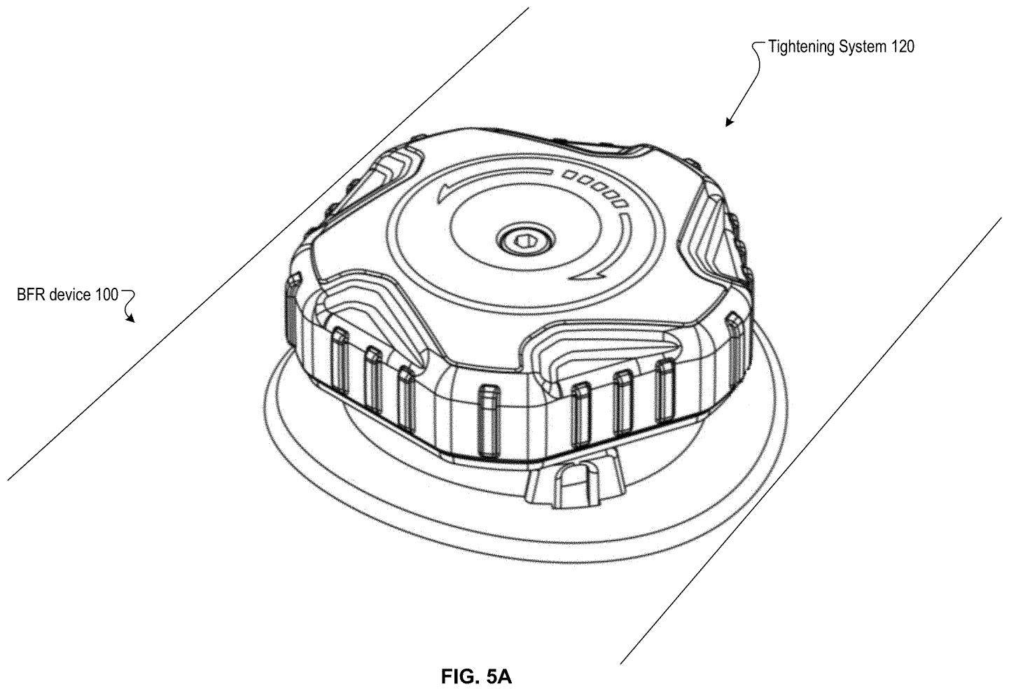

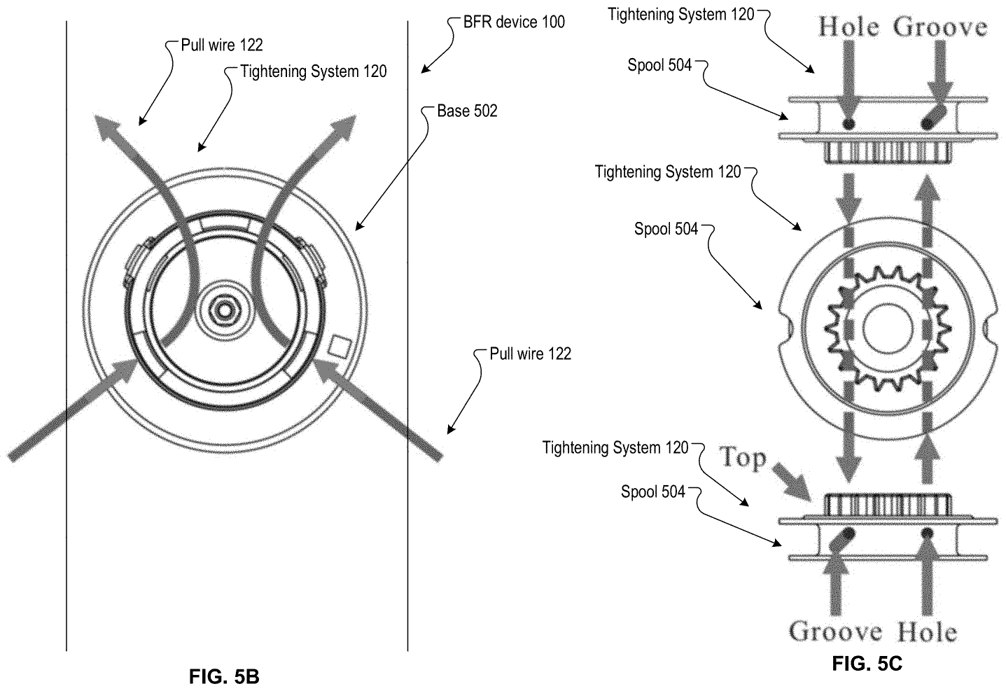

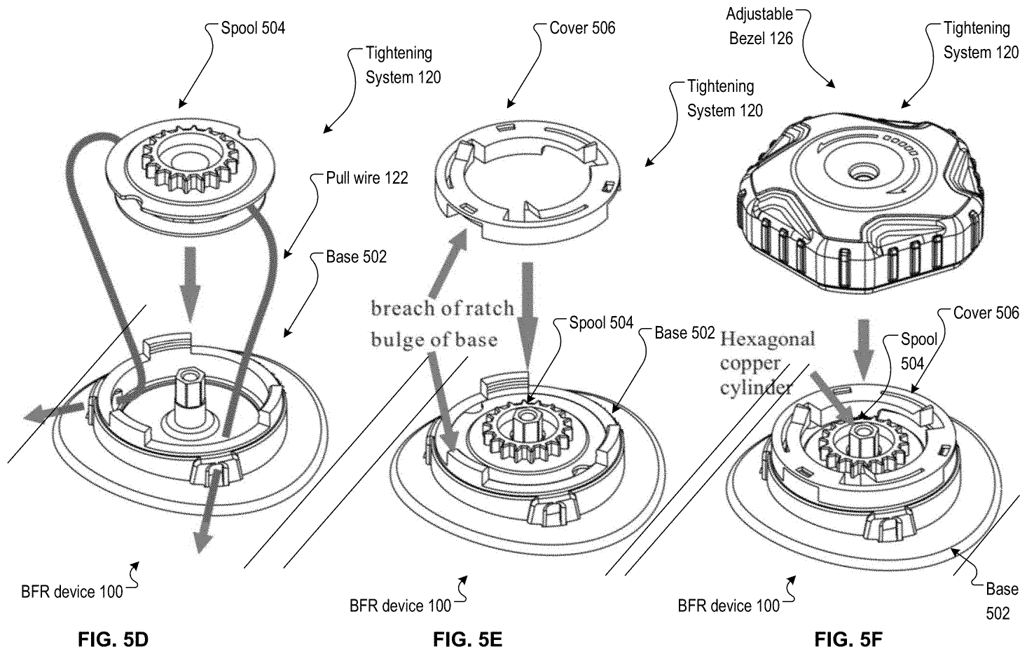

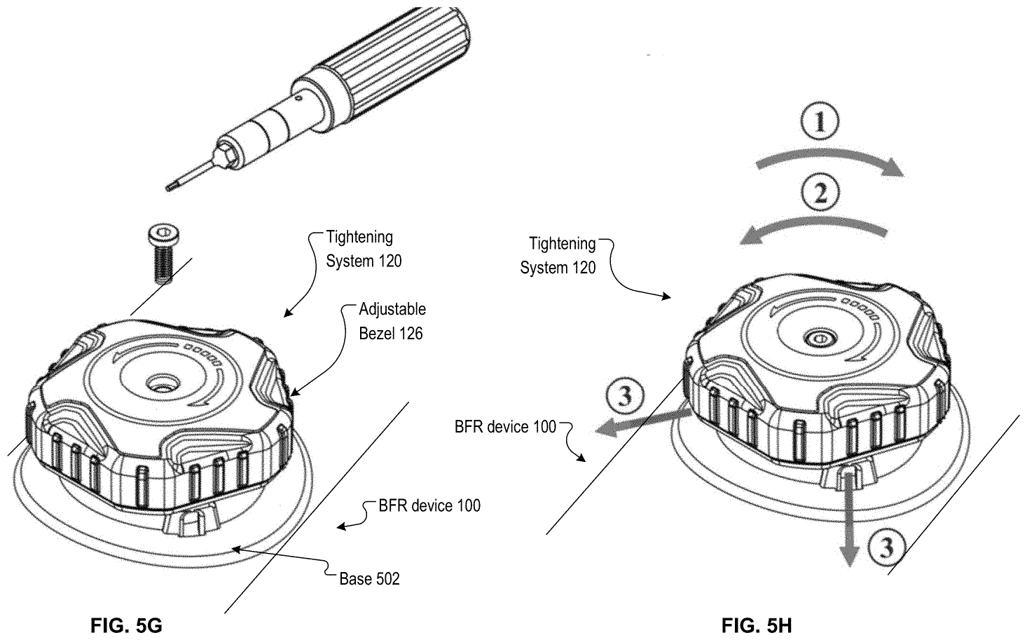

The BFR device 100 includes a tightening system 120. The tightening system 120 includes a pull wire 122 (e.g., a steel wire lace) disposed within the cuff 110 (e.g., within the outer structure 114), a spool (see FIGS. 5C-F) disposed within the cuff 110 and connected to a first distal end of the pull wire 122, a rivet 124 connected to a second distal end of the pull wire 122, and an bezel 126 (e.g., dial) connected to the spool for tightening and loosening of the cuff 110 via rotation of the bezel 126. In some embodiments, a distal end of the pull wire 122 is routed around the rivet 124 and is coupled to the pull wire 122 (e.g., via crimping, via an anchor, etc.). In some embodiments, the pull wire 122 is routed through the rivet 124 (e.g., through an opening formed by the rivet 124) and is configured to not be removed from the rivet 124 (e.g., via crimping, via an anchor, via connection with a portion of the pull wire 122).

The pull wire 122 may have a pull length determined by the amount of occlusion desired. In the injection molded embodiment, the receptor end of the tightening may be incorporated into the injection molding process.

The tightening system 120 may include a base attached to the interior structure 112, a spool disposed on the base, and a bezel 126 (e.g., adjustable bezel) attached to the spool and/or base (e.g., the spool is disposed between the base and the bezel 126). The rivet may be attached to the interior structure 112.

The tightening system 120 may further include a hanger 128 (e.g., lace guide) that is disposed in the cuff 110 (e.g., within the outer structure, connected to the cuff 110 via the pull wire 122). The pull wire 122 may pass through the hanger 128 so that responsive to rotating the bezel 126, the pull wire 122 retracts and pulls the hanger 128 (e.g., and the strap 150 connected to the hanger 128) from the full extension position towards the full retraction position. In some embodiments, the rivet 124 may extend from the outer surface of the cuff 110 into the cuff 110 (e.g., passes through the outer structure 114 and the interior structure 112 of the cuff 110). In some embodiments, the rivet 124 is attached to the interior structure 112 of the cuff 110 without passing through the outer structure 114 of the cuff 110. The tightening system 120 (e.g., bezel 126) may include a face (e.g., circumscribed by the outer portion of the bezel 126) that remains stationary when the spool and the outer portion of the bezel 126 are rotating or rotated to set to zero. Responsive to circumscribing an arm, the bezel 126 may be on an outer portion of the cuff 110 on the inside of the arm to be viewable and rotatable by the user.

In some embodiments, the tightening system 120 may be a closure system. For example, the tightening system 120 may be a ATOP closure system (e.g., A-B15) or Boa® closure system (e.g., Boa® H4 dial, Boa® H2 dial, and the like). The bezel 126 may be accessible at an outer surface of the cuff 110. The bezel 126 may have ridges to allow rotation of the bezel 126. The ridges may allow the bezel 126 to be rotated by a user by hand. The ridges may allow a small motor to rotate the bezel 126 for remote setting that activates the spool (such as in a mobile application (an app)). Rotation of the bezel 126 may rotate the spool, causing the pull wire 122 to wrap around the spool and tighten the cuff 110. Rotation of the bezel 126 may cause the cuff 110 to go from a full extension to a full retraction (as shown in FIG. 1). Pulling the bezel 126 away from the outer surface of the cuff 110 may unlock the spool and allow the pull wire 122 to unwind from the spool (e.g., to return the cuff 110 to a full extension position).

As shown in FIG. 1C, the bezel 126 may include indication of tightness of the BFR device 100. A face of the tightening system 120 (e.g., face of spool, face of bezel 126) may stay stationary (e.g., at all times, while the bezel 126 rotates) or the bezel 126 may rotate to reset to the spool. The face of the tightening system 120 (e.g., spool) may include a first marking (e.g., a dot) and the bezel 126 may include a second marking (e.g., a dot, color, percentage, value, and the like). A user may align the first marking and the second marking for a predetermined tightness (e.g., 20%, 40%, 60%, or 80% tightness). The bezel 126 may be a circular measurement meter that allows the user to find and return to consistent tightness readings (e.g., a value shown on the bezel 126) which may be more accurate than reading air-pressure in a surgical cuff.

In some embodiments, the bezel 126 may be unlocked by pulling the bezel 126 away from the spool and the bezel 126 may be rotated to line up an indicator (e.g., dot) on the bezel and an indicator on the face of the spool. After being rotated to line up the indicator (e.g., and the hanger being pulled to a fully extended position), the bezel 126 may be locked by pressing the bezel 126 towards the spool. In some embodiments, the upper surface of cuff 110 may form a slit window and an indicator (e.g., an arrow) may slide along the slit as the tightening system 120 is tightened or loosened.

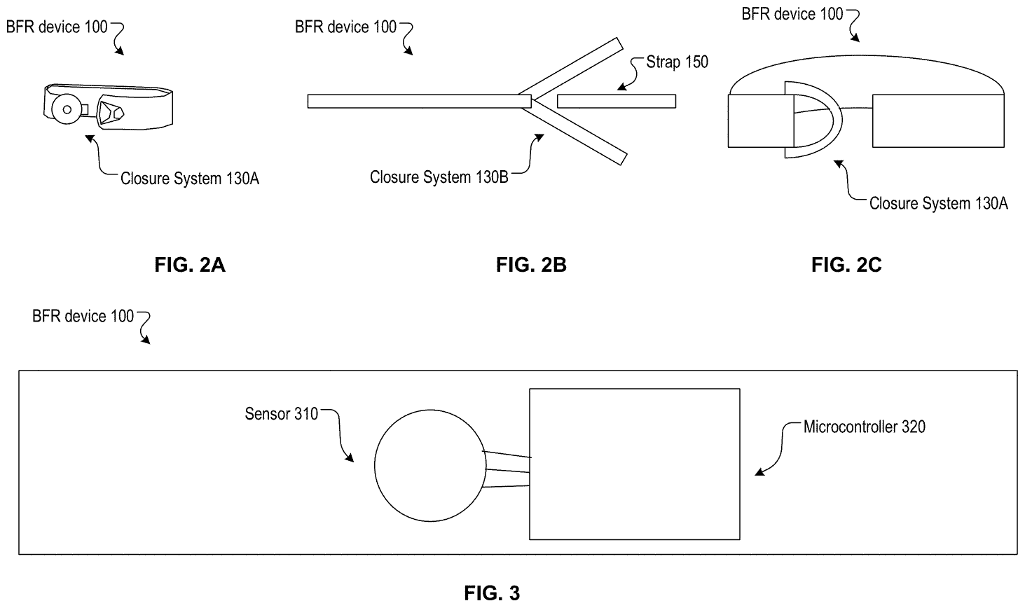

Returning to FIG. 1A, the BFR device 100 may include a closure system 130. The closure system 130 may include a hoop-and-loop fastener (e.g., Velcro®). The closure system 130 may be one or more of a quick-fit strap closure (see FIG. 2A), an alligator fit closure (see FIG. 2B), a D-clip closure (see FIG. 2C, disposed on the outer surface of the cuff 110 to minimize pinching of the skin of the user), a snap button fixed extension length adjustments, variable lengths of material connected by a snap or by a magnetic closure system, a pull-in strap, and/or the like. For example, a strap 150 (e.g., pull-in strap) may removably attach to an alligator fit closure to secure the BFR device 100 around a portion of a user. The closure system 130 (e.g., pull-in strap) may be trimmed to fit to the portion of the body of the user (e.g., to customize size of the BFR device 100 creating an enclosed cuff instead of an adjustable self-tightened strap). In some embodiments, the pull wire 122 may be fully extended and the pull-in strap (e.g., distal cuff end) may cut to a length where responsive to closing the closure system 130 (e.g., attaching the pull-in strap to the alligator fit closure), the BFR device 100 circumscribes the portion of the body (e.g., fits around the portion of the body so that rotation of the bezel 126 causes pressure to be applied to the portion of the body). A unique marking may be made on the strap 150 to indicate a partial retraction of the strap 150 that allows users who have customized the cuff, to easily remove and reinstall the cuff on the extremity without the need to open the closure system 130 or re-thread the “D-ring” (e.g., that connects the pull-in strap to the cuff 110).

The closure system 130 may be used for an initial tightness that allows the BFR device 100 to be put on and taken off without loosening the closure system 130. The tightening system 120 may provide further tightening for BFR, e.g., partial occlusion of the arterial flow and up to total occlusion of the venous flow, and loosening to remove the BFR. The closure system 130 may not provide BFR when the tightening system is in a full extension position.

The BFR device 100 may be different sizes for different sizes of users. In some embodiments, the closure system 130 at a first distal end of the cuff 110 (e.g., the alligator fit closure) is 2 inches long, the closure system 130 at a second distal end of the cuff 110 (e.g., a pull-in strap) is 10 inches long, a portion of the cuff 110 between the pull wire 122 at full extension and the pull-in strap may be 1 inch, the difference in length of the cuff between the full extension of the pull wire 122 and the full retraction of the pull wire 122 may be 5 inches, and the distance between the closure system 130 at the first distal end of the cuff 110 and the pull wire 122 at the full retraction may be 2 inches. A BFR device 100 for arms may have a width of 5 centimeters (cm). A BFR device 100 for legs may have a width of 10 cm. A BFR device 100 may have a width of 15 cm for larger legs.

The BFR device 100 may have a speed setting for easy on/off that indicates an amount of pull up strap that remains available for loosening. The BFR device 100 may indicate an area that is one inch from full retraction of the BOA, so when fastened initially, the BFR device 100 may be put on, tightened, and then loosened without adjusting the closure system 130.

The outer structure 114 (e.g., fabric sleeve) of the cuff 110 may be a material that can receive a dye sub transfer to personalize and brand the BFR device 100 (e.g., personalized with one or more of images, logos, or words).

In some embodiments, the strap 150 includes one or more of a nylon seat belt webbing material, leather, hook material, loop material, and/or the like.

FIG. 1B illustrates a cross-sectional side view of a BFR device 100, according to certain embodiments. BFR device 100 of FIG. 1B may be the same or similar to BFR device 100 of FIG. 1A.

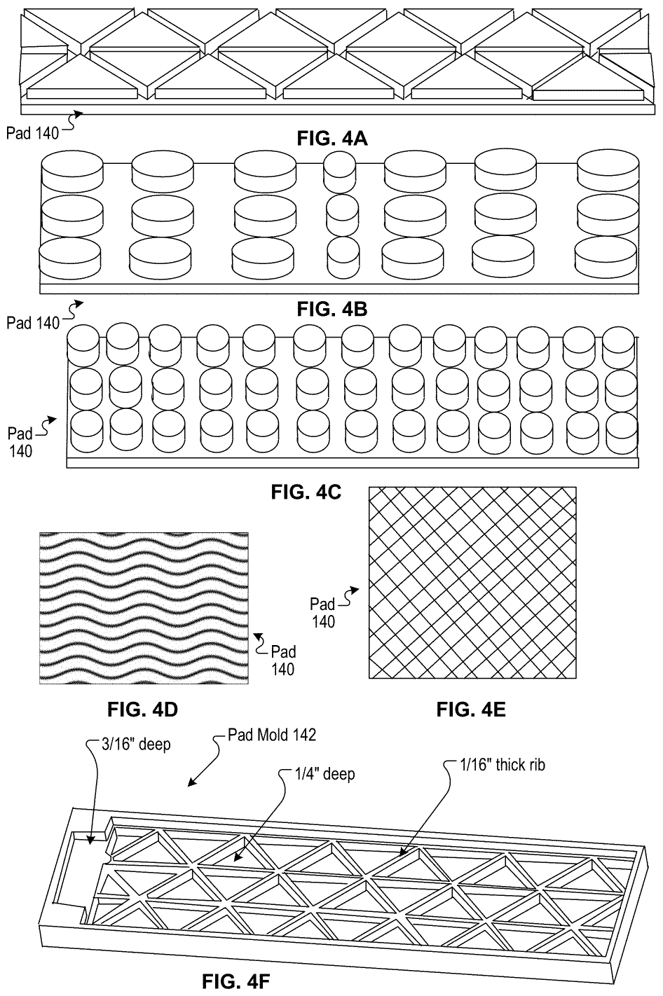

The BFR device 100 may include a pad 140 (e.g., cushion, silicon-based cushion, etc.) disposed on an inner surface of the cuff 110. The pad 140 may be a silicon-based cushion. In some embodiments, the pad 140 is one or more of a platinum-based silicon material, platinum-infusion silicon cushion, a platinum-cure silicone, a tin-cure silicone, a silicon rubber, silicone casting, etc. The pad 140 may be for partially occluding arterial flow (e.g., of a portion of a body, such as an arm, leg, etc.) while substantially (e.g., completely) occluding venous flow. The pad 140 may be made of a material that has a variable durometer using designs (see FIGS. 4A-E) that maximize compression effectiveness without fully occluding arterial flow. The pad 140 may allow the BFR device 100 to approximate the effect of air bladder cuffs (e.g., and their derivatives) without the need for inflation or real-time monitoring. The pad 140 may allow for different body mass index (BMI) of users. The pad 140 may be secured to the cuff 110 (e.g., via elastic bands, via adhesive, etc.). The pad 140 may be removable from the cuff 110 and interchangeable with other types (e.g., type of material, different material of durometer, different type of design) and sizes of pad 140. The pad 140 may include a quick release from the inside surface of the cuff 110 for one or more of hygiene, cleaning, or use of another pad 140 (e.g., with a different durometer). In some embodiments, the cuff 110 has connecting components (e.g., elastic bands (e.g., elastic straps) that are configured to secure the pad 140 to the cuff 110).

The pad 140 may include one or more designs with cuts (e.g., indentations, ridges, channels formed by extrusions, etc.) in the outer surface of the pad 140 to allow the material to compress with variable “hard and soft” areas. In some embodiments, the design (e.g., ridges and grooves) of the pad 140 may be one or more of ravioli, air bladder, waves, reverse waffle, waffle, ebelskiver, etc. The pad 140 may include a material used for yoga mats, exercise mats, or work mats. The pad 140 may be a material including one or more of polyvinyl chloride (PVC), natural rubber, recycled rubber, thermoplastic elastomers (TPE), jute fiber, polymer environmental resin (PER), ethylene-vinyl acetate (EVA), nitrile rubber (NBR), polyurethane (PU), expanded polyethylene (EPE foam), etc.

The pad 140 may have a variable cushion durometer that are advantageous for use on variable user BMI characteristics and for type of compression (e.g., 80% vs. 20%). In some embodiments, the pad 140 is soft like gummy bears (e.g., about 10 on shore 00 hardness scale) for low occlusion rates and low BMI users. In some embodiments, the pad 140 is hard like rubber band or soft eraser (e.g., about 60-80 on shore 00 hardness scale, about 20-40 on shore A hardness scale) for high BMI and performance and hypertrophy users. In some embodiments, the pad 140 has a value of 3-80 on the shore 00 hardness scale. In some embodiments, the pad 140 has a value of 3-30 on the shore 00 hardness scale. In some embodiments, the pad 140 has a value of 10-30 or greater on the shore 00 hardness scale.

In some embodiments, the BFR device 100 may partially occlude arterial flow between 20% and 80% and may totally occlude venous flow.

In some embodiments, the pad 140 is about one fourth of an inch to three eights of an inch in thickness. The BFR device 100 may include an interior structure 112 that may be a malleable plastic shield under the wires (e.g., under pull wire 122). The interior structure 112 may be disposed under the pull wire 122 and may extend about one half inch longer (e.g., towards the pull-in strap) than the hanger 128.

FIGS. 2A-C illustrate closure systems 130 for BFR device 100, according to certain embodiments. In some embodiments, a first closure system 130 (e.g., a first type of closure system 130) is disposed at a first distal end of the cuff 110 and a second closure system 130 (e.g., a second type of closure system 130 that is different from the first type of closure system) is disposed at the second distal end of the cuff 110. In some embodiments, one or more of the closure systems 130 of the BFR device 100 removably attaches the cuff 110 to the strap 150. In some embodiments, a first portion of the closure system 130 (e.g., a first closure system 130) removably attaches a first distal end of the cuff 110 to the strap 150 and a second portion of the closure system 130 (e.g., a second closure system 130) attaches the second distal end of the cuff 110 to the strap 150.

FIG. 2A illustrates a closure system 130A that is a quick-fit strap closure. The quick-fit strap closure may include a connector after strap length adjustment.

FIG. 2B illustrates a closure system 130B that is an alligator fit closure. In some embodiments, the quick-fit strap closure may adhere to the alligator fit closure and then the strap 150 (e.g., pull-in strap of FIG. 1A) may be cut and fit into the alligator fit closure.

In some embodiments, the alligator fit closure includes hooks (e.g., first side of Velcro®) on a first interior surface and loops (e.g., second side of Velcro®) on a second interior surface and the pull-in strap includes loops on a first exterior surface and hooks on a second exterior surface. The pull-in strap may have soft material around the edges to minimize abrasiveness of the hook-and-loop material. The alligator fit closure may be opened and the pull-in-strap may be disposed within the alligator fit closure so that the hooks of the first interior surface attaches to the loops of the first exterior surface and the loops of the second interior surface attach to the hooks of the second exterior surface.

FIG. 2C illustrates a closure system 130A that is a D-clip closure. The strap 150 may be pulled through the D-clip closure, cut, and attached to hooks or loops surface of the strap 150 (e.g., via a connection component 152).

FIG. 3 illustrates a BFR device 100 including one or more sensors 310 and a microcontroller 320, according to certain embodiments. The BFR device 100 may include two or more sensors 310, where two or more of the sensors have different functionalities. The sensor 310 may provide a reading via one or more of a user interface, a light emitting diode (LED), audio alert, wireless transmission, etc. The one or more sensors 310 may include one or more of a sensor to detect repetitions, a sensor to detect time in use, a sensor to detect venous push, a sensor to detect occlusion percentage based on blood volume, and the like. The BFR device 100 may include one or more of a time with cadence, an audio source for music, storage of a hygienic spray, and the like. The BFR device 100 may include or be associated with a perfusion index (PI) measurement device (e.g., finger pulse rate of oxygen consumption (VO2) measurement, maximal oxygen uptake (VO2 max)) as a predictor of relative tightness.

The sensor 310 may be an LOP sensor for a BFR device to provide an indication of an LOP reading. The BFR device 100 may include a pocket or slot for the sensor 310 on the occlusion side of the limb to detect artery activity with an LED indicator. The LOP sensor may be a surgical research monitoring sensor to show visible evidence of LOP activity using an LED on the occluded limb side of the BFR device 100 (e.g., on the inside of the arm). The LOP sensor may be located on the occluded side of the limb downwind from the occlusion.

On ore more of the sensors 310 may contact the skin of the user, e.g., extend through the pad 140, extend beside the pad 140, extend from an inner surface of the cuff 110 that is not covered by the pad 140, and the like.

The BFR device 100 may further include a microcontroller 320. The microcontroller 320 may be coupled to one or more of the sensors 310. The microcontroller 320 may be coupled to one or more electronic devices (e.g., sensor, speaker, LED, memory, music playing device, battery, wireless transmitter, and the like). The microcontroller 320 may receive sensor data from one or more sensors 310 and cause the sensor data to be transmitted and/or displayed.

FIGS. 4A-E illustrate pads 140 for a BFR device 100, according to certain embodiments. A pad 140 may include one or more of indentations, ridges, grooves, cuts, extrusions, channels, and the like in an outer surface that is to be placed opposite of the inner surface of the cuff 110. In some embodiments, the outer surface of the pad 140 is to be in contact with the skin of the user. Responsive to the BFR device 100 being tightened (e.g., via the tightening system 120), the pad 140 may compress, e.g., the ridges may come together at the channels, cuts, or grooves. FIG. 4A illustrates a ravioli design. FIGS. 4B-4C illustrate ice-cube tray designs. FIG. 4D illustrates a wavy design. FIG. 4E illustrates a waffle design. Each pad 140 (e.g., silicon-based cushion) may form ridges and recesses configured to compress together responsive to tightening of the BFR device 100.

FIG. 4F illustrates a mold 142 for a pad 140 for a BFR device 100, according to certain embodiments. In some embodiments, the mold 142 produces pads 140 with recesses of about 1/16 inch thickness (e.g., the mold has ribs that are about 1/16 inch thick). In some embodiments, the mold 142 forms extrusions (e.g., triangular extrusions) that are about ¼ inch tall (e.g., the pad mold 142 has recesses that are about ¼ inch deep). In some embodiments, the pad mold 142 forms a pad with a distal end that is about 3/16 inch tall (e.g., the pad mold 142 has a recess at a distal end that is 3/16 inch deep). The pad 140 may be generated by placing material in the pad mold 142 for a threshold amount of time (e.g., one hour). Different designs on the pad 140 may be generated by placing a design (e.g., drawing, letters, numbers, etc.) inside the pad mold 142.

FIGS. 5A-H illustrate tightening systems 120 of BFR devices 100, according to certain embodiments. One or more of FIGS. 5A-H may display the same tightening system 120 (e.g., from a different view, exploded or assembled view). FIG. 5A is a perspective view of a tightening system 120 of a BFR device 100, according to certain embodiments.

FIG. 5B is a top view of a tightening system 120 of a BFR device 100, according to certain embodiments. The tightening system 120 includes a base 502 and pull wire 122 (e.g., wire lace). The pull wire 122 may be threaded into holes of the base 502.

FIG. 5C is a top view, front view, and back view of the spool 504 (e.g., reel) to be disposed on the base 502 of BFR device 100 (e.g., of FIG. 5B). The spool 504 may include one or more holes and may include one or more grooves for the pull wire 122. In some embodiments, the spool 504 includes a hole and a groove on a front surface of the spool 504 and the spool 504 includes a hole and a groove on a back surface of the spool 504. Each groove may be substantially opposite a hole. In some embodiments, each groove includes two holes. A first distal end of the pull wire 122 may be threaded through the hole on the front of the spool 504 into the interior of the spool 504, through a first hole of the groove on the back of the spool 504 to the exterior of the spool 504, and then through a second hole of the groove on the back of the spool 504 to the interior of the spool 504 (forming a loop). The pull wire 122 may be pulled until the loop of the pull wire 122 is fully fixed into the groove. The pull wire 122 threaded through the hole on the front and the holes in the groove on the back may secure pull wire 122 to the spool 504. In some embodiments, a second distal end of the pull wire 122 is secured to spool 504 by being threaded through the hole on the back of the spool 504 and the groove on the front of the spool 504 in a similar manner. The pull wire 122 may be secured to hanger 128 so that rotation of the spool 504 wraps the pull wire 122 around the spool 504 and moves the hanger 128 closer to the spool 504 (e.g., from full extension towards the full retraction).

In some embodiments, a first distal end of a first pull wire 122A is attached to the spool 504 as described above, a second distal end of the first pull wire 122A is attached to the hanger 128, a first distal end of a second pull wire 122B is attached to the spool 504, and a second distal end of the second pull wire 122B is attached to the hanger 128. The rotating of the spool 504 may wrap the first pull wire 122A and second pull wire 122B around the spool 504 (and rotation in the opposite direction may unwrap the first pull wire 122A and the second pull wire 122B from around the spool 504).

FIGS. 5D-F illustrate exploded perspective views of tightening systems 120 of BFR devices 100, according to certain embodiments. As illustrated in FIG. 5D, the tightening system 120 may include a base 502, spool 504 (e.g., reel), and pull wire 122. The pull wire 122 may be pulled toward the exterior of the base 502 to pull the spool 504 into the base 502.

As illustrated in FIG. 5E, the tightening system 120 may include a base 502, spool 504, and a cover 506 (e.g., ratch). A breach (e.g., groove) formed by the cover 506 may be placed in an extrusion (e.g., bulge) of base 502.

As illustrated in FIG. 5F, the tightening system 120 may include a base 502, spool 504, a cover 506, and bezel 126 (e.g., knob). An interior surface of the bezel 126 may form a hole (e.g., hexagonal hole) that matches an extrusion (e.g., hexagonal copper cylinder) of the base 502 for coupling the bezel 126 to the base 502.

The base 502 may be disposed in or attached to the cuff 110. In some embodiments, the base 502 is attached to interior structure 112 (e.g., sewn to interior structure 112). The outer structure 114 may form an opening (e.g., a hole in the fabric) and at least a portion of the tightening system 120 (e.g., the bezel 126) may extend through the outer structure 114. The spool 504 may be disposed on the base 502, the cover 506 may be disposed on the spool 504, and the bezel 126 may be disposed on the cover 506. The pull wire 122 may go through a first opening in the base 502, through or around the spool 504, and through a second opening in the base 502. The cover 506 may form a slot (e.g., a breach of ratch) that couples with an extrusion (e.g., a bulge) of the base 502. The base may include a hexagonal copper cylinder that is to go through the spool 504 and the cover 506 to couple with a fastening component (e.g., screw) to secure the bezel 126 to the base 502.

FIG. 5G is a perspective view of a tightening system 120 of a BFR device 100, according to certain embodiments. A fastening component (e.g., screw) may attach the adjusting bezel 126 to the base 502. FIG. 5H is a perspective view of a tightening system 120 of a BFR device 100, according to certain embodiments. The adjusting bezel 126 may be rotated clockwise to tighten the pull wire 122 (e.g., pull the hanger 128 closer to the base 502) and the adjusting bezel 126 may be rotated counter-clockwise to release the pull wire 122 (e.g., allow the hanger 128 to move further away from the base 502).

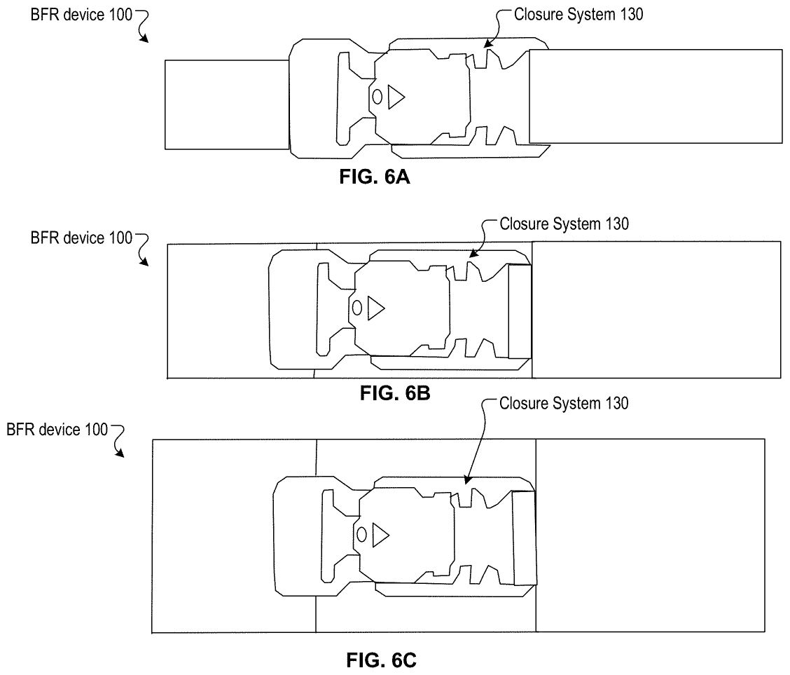

FIGS. 6A-C illustrate closure systems 130 (e.g., clips, buckles, etc.) for BFR devices 100, according to certain embodiments. As shown in FIGS. 6A-C, the cuff 110 and/or strap 150 may be different widths for use by different users or to be used on different parts of the body, e.g., larger width for larger user and smaller width for smaller user, larger width for leg and smaller width for arm. The closure systems 130 may be coupled to one or more of a strap 150 and/or the cuff 110.

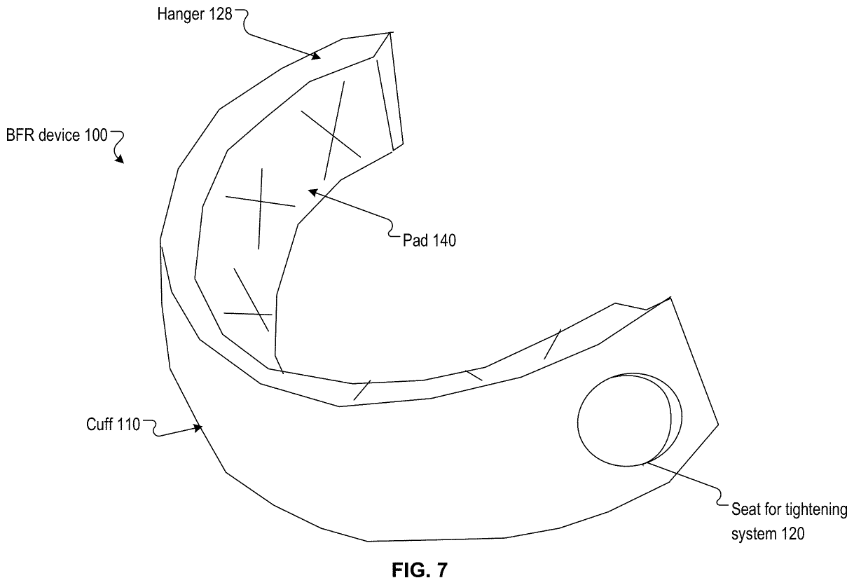

FIG. 7 illustrates a BFR device 100, according to certain embodiments. In some embodiments, FIG. 7 illustrates a BFR device 100 formed by injection molding (e.g., an injection molded plastic). The BFR device 100 may include a cuff 110. The cuff 110 may be made of a semi-rigid plastic. The BFR device 100 may include a pad 140 (e.g., cushion layer). In some embodiments, the pad 140 is removably attached to the cuff 110 (e.g., via elastic bands). In some embodiments, the pad 140 is non-removably attached to the cuff 110. In some embodiments, the pad 140 is integral to the cuff 110. The BFR device 100 may include a seat (e.g., in the cuff 110) for the tightening system 120. A hanger 128 may be disposed in the cuff 110 proximate a first distal end of the cuff 110 and the seat for the tightening system 120 may be disposed proximate a second distal end of the cuff 110.

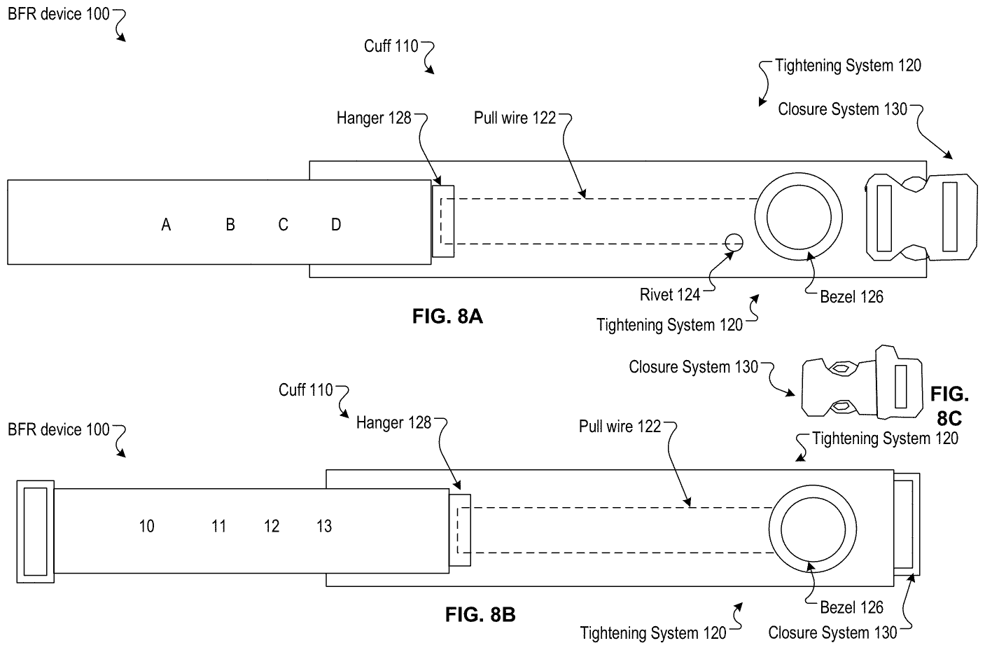

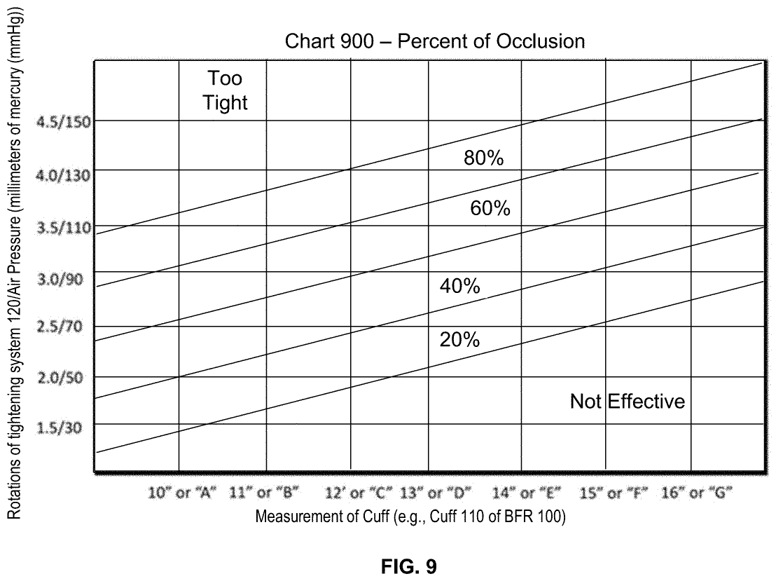

FIGS. 8A-C and 9 illustrate a system (e.g., that could be used as a Heart Rate Training Guide) that allows users of BFR devices 100 (e.g., of FIGS. 8A-B) to consult with a chart (e.g., chart 900 of FIG. 9) to determine the level of tightness or pressure in the cuff 110 of the BFR device 100.

FIGS. 8A-C illustrate one or more BFR devices 100, according to certain embodiments. Each BFR device 100 includes a cuff 110, a closure system 130, and a tightening system 120. The tightening system 120 may include a rivet 124, a pull wire 122, and an bezel 126 (e.g., coupled to a spool where rotation of the bezel 126 cause the pull wire 122 to wind around the spool or unwind from the spool). The closure system 130 may include a buckle with a slot for a distal end of the cuff 110 to be inserted through the slot and attached to itself (e.g., via a hoop-and-loop fastener, such as Velcro®). The buckle may be unbuckled for quick removable of the BFR device 100 from a limb. In some embodiments, the closure system 130 does not include a buckle and the BFR device 100 is removed by adjustment (e.g., rotating, pulling out, etc.) of the tightening system 120. In some embodiments, the tightening system 120 does not include a rivet 124 and both ends of the pull wire 122 are attached to the spool of the tightening system 120 (e.g., rotation of the bezel 126 wraps both ends of the pull wire around the spool, see FIG. 8B). In some embodiments, the bezel 126 is a grooved ring. In other embodiments, the bezel 126 is an upper surface that covers other components of the tightening system 120 (e.g., covers cover 506 and spool 504 of FIG. 5F). In some embodiments, the closure system 130 (e.g., including a buckle and a slot of a smaller width) of FIG. 8C may be used.

In some embodiments, the cuff 110 includes a set of markings that indicate a starting measurement of the cuff 110 responsive to the cuff 110 being snug (e.g., tightened around limb without occluding arterial flow) on the limb of the user. The markings could include one or more of numbers, letters, or markings that correspond to actual circumference of the limb. The width of a cuff may be a determinate factor in safe occlusion. The law of LaPlace indicates tension equals pressure times the radius (t=P*r). In some embodiments, the retractable strap of the cuff 110 may be marked with the letters “A,” “B,” and so on that corresponding to actual measurements of 10 inches =“A,” 11 inches =“B,” and so on as shown in FIGS. 8A-B. The user may reference a chart that includes a published scale (e.g., chart 900 FIG. 9). For example, if the BFR device 100 is tight (e.g., not occluding arterial blood flow, not falling off limb) at 10 inches or marking “A,” that may relate to about 1.5 rotations of the tightening system 120 which may be about 30 millimeters of mercury (mm Hg). Then, at 2 rotations (e.g., of the tightening system 120 of the BFR device 100) may correspond to 20% occlusion of arterial flow while occluding venous flow, 2.5 rotations to 40%, 3.0 rotations to 60%, 3.5 rotations to 80%, and 4.0 and above may be too tight (e.g., occluding more than 80% arterial flow may lead to injury). In another example, if BFR device 100 is tight (e.g., not occluding arterial blood flow, not falling off limb) at 15 inches or marking “F” that may relate to 2.5 rotations, 3.0 rotations (of tightening system 120) may correspond to 20% (occlusion of arterial flow), 3.5 rotations to 40%, 4.0 rotations to 60%, 4.5 rotations to 80%, and 5.0 rotations may be too tight.

As the BFR device 100 (e.g., cuff 110) is tightened (e.g., via user input of rotating the tightening system 120), the number of clicks or dial rotations of the tightening system 120 may correspond to a specific measure of retraction of the strap which in turn relates to the actual percentage of reduction in the circumference of the cuff. For example, the overall cuff length is retracted 2 inches which equals 72 clicks (1mm per click) or 3 full rotations of the dial (tightening system 120), the overall circumference of the cuff 110 may be reduced from 15 inches to 13 inches (e.g., a reduction of 13%). Reducing the circumference by 13% applies “x” pressure. Reducing a 12 inch limb by 2 inches is a 16.6% reduction of circumference, so the relative tightness or pressure of the same amount of rotations is more for a smaller limb.

The amount of circumference reduction also creates a measurable pressure in terms of pounds per square inch (psi). The pressure may be dependent on the initial measurement of the limb which may be taken into account by the chart on FIG. 9.

FIG. 9 illustrates the percent of occlusion in a chart 900, according to certain embodiments. On a pneumatic cuff, the pressure indicated in the air bladder may correspond to a level of pressure that the cuff puts on the skin of the user. The chart of FIG. 9 may be used for multiple types of cuffs, including pneumatic (air bladder) and non-pneumatic (cuff 110 of BFR device 100).

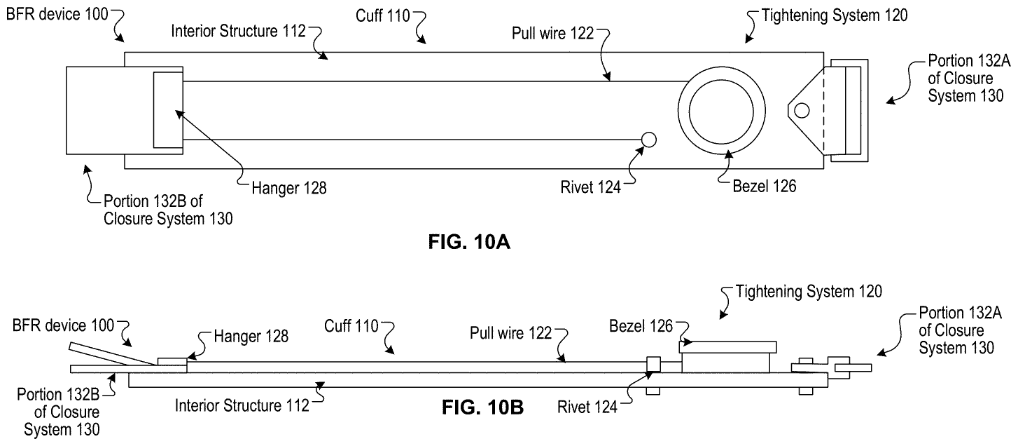

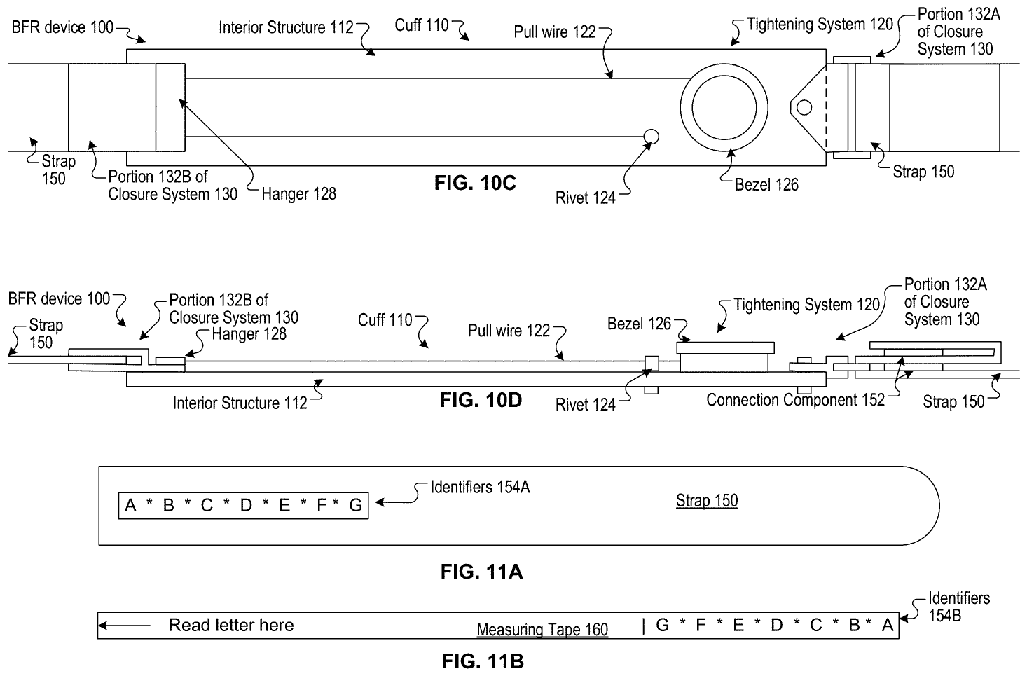

FIGS. 10A-D illustrate BFR devices 100, according to certain embodiments. In some embodiments, a BFR device 100 includes a cuff 110 and a strap 150. In some embodiments, the cuff 110 and strap 150 are permanently attached to each other (e.g., non-removably attached, integral to each other). In some embodiments, the cuff 110 and strap 150 are configured to removably attach to each other (e.g., the cuff 110 and the strap are two distinct components). FIGS. 10A-B illustrate a cuff 110. FIGS. 10C-D illustrate a cuff removably attached to a strap 150. FIGS. 10A-D illustrate the interior structure 112 without the outer structure 114 (e.g., fabric sleeve) and without pad 140 for clarity of components of the BFR devices 100. An outer structure 114 may be disposed over the interior structure 112 and a pad 140 may be attached to the outer structure 114. The outer structure 114 may be sized to cover the pull wire 122, rivet 124, and hanger 128. The outer structure 114 may have an opening to expose the bezel 126.

In some embodiments, the closure system 130 includes a portion 132A (e.g., D-ring at a first distal end of the cuff 110) of the closure system 130, a portion 132B (e.g., alligator fit closure at a second distal end of the cuff 110) of the closure system 130, and strap 150 (e.g., that has a first distal end that removably attaches to the portion 132A and that has a second portion that removably attaches to the portion 132B).

A portion of the tightening system 120 (e.g., base 502), the rivet 124, and the portion 132A of the closure system 130 may be attached (e.g., non-removably attached) to the interior structure 112. The hanger 128 may be attached (e.g., non-removably attached) to the portion 132B (e.g., alligator fit closure) of the closure system 130. The pull wire 122 may be routed through the hanger 128. Responsive to rotating the bezel 126, the pull wire 122 may become wrapped within the tightening system 120 (e.g., around spool 504) which may pull the hanger 128 and portion 132B of the closure system 130 towards the bezel 126. Pulling the portion 132B of the closure system 130 may cause the portion 132B of the closure system 130 and a portion of the strap 150 to be pulled within the outer structure 114 of the cuff 110. The interior structure 112 may have a smooth upper surface to allow the portion of the closure system 130, strap 150, hanger 128, and/or pull wire 122 to be pulled across the upper surface of the interior structure 112.

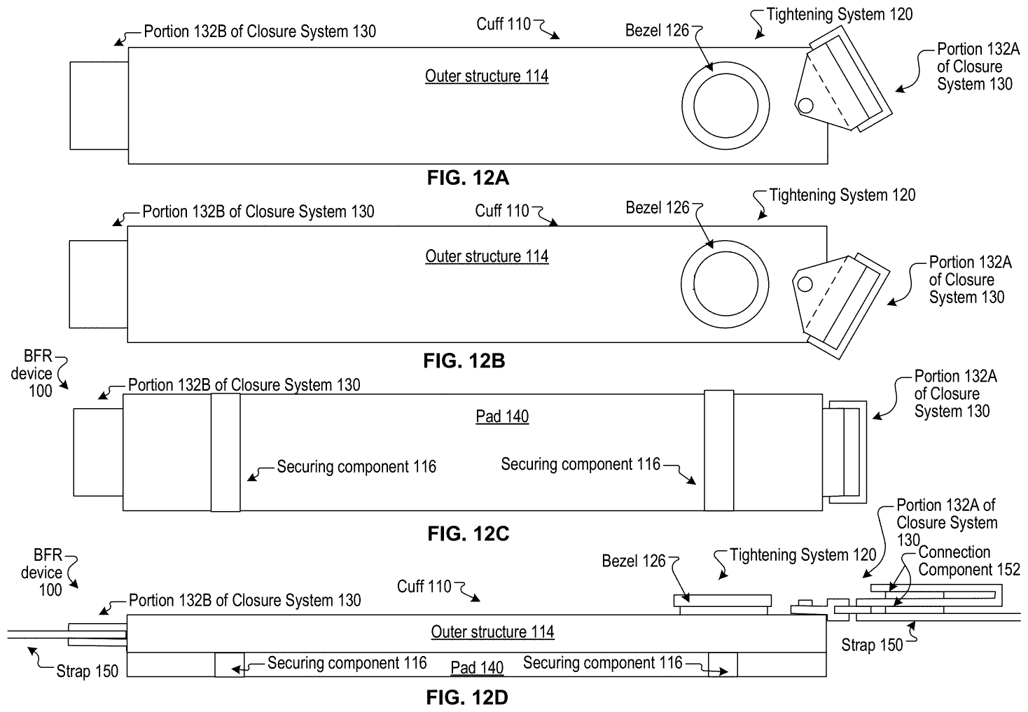

The portion 132A of closure system 130 may be attached to the interior structure 112 (e.g., and at least a portion of the outer structure 114) via a pivot point (e.g., a rivet with washers under the interior structure 112, etc.). The portion 132A of the closure system 130 may be a D-clip and may pivot about the pivot point as illustrated in FIGS. 12A-B to provide for fine adjustment of the cuff 110 around limbs for comfort (e.g., the D-ring may provide a contour feature). This may allow for positioning the BFR device 100 on different limbs, at different locations on the same limb, on different users, and/or the like. For example, portion 132A of the closure system 130 may pivot to a first orientation (e.g., a first contour) around the upper-most portion of the leg and may pivot to a second orientation (e.g., a second contour) around a lower or mid portion of the thigh.

The strap 150 may include a first portion configured to removably attach to a portion 132A of closure system 130 (e.g., a first distal cuff end of the cuff 110) and a second portion configured to removably attach to a portion 132B of closure system 130 (e.g., second distal cuff end of the cuff 110).

The portion 132A of closure system 130 include a D-ring rotatably attached to the cuff 110. A portion of the strap 150 is configured to be routed through the D-ring and removably attached to another portion of the strap 150 via a connection component 152.

The portion 132B of closure system 130 may include an alligator fit closure that includes an upper alligator portion and a lower alligator portion. To attach the first portion to the alligator fit closure, a lower strap surface of a portion of the strap 150 is to be disposed on an upper surface of the lower alligator portion and a lower surface of the upper alligator portion is to be disposed on an upper strap surface the portion of the strap 150.

The strap 150 may have an upper strap surface and a lower strap surface that include a first type of material (e.g., loop material) configured to secure to a second type of material (e.g., hook material). The upper surface of the lower alligator portion and the lower surface of the upper alligator portion may include the second type of material (e.g., hook material). The upper and lower surfaces of the connection component may include the second type of material (e.g., hook material).

FIG. 11A illustrates the strap 150, according to certain embodiments. The strap 150 may have a first distal end that is a finished end (e.g., rounded end). The strap 150 may have a second distal end that has one or more identifiers 154A (e.g., markings, letters, applied to the strap 150 via removable tape).

FIG. 11B illustrates a measuring tape 160, according to certain embodiments. The measuring tape 160 may have one or more identifiers 154B (e.g., markings, letters, etc.) at a first distal end of the measuring tape 160. A second distal end of the measuring tape 160 may indicate to identify the identifier at that second distal end (e.g., “read letter here,” etc.). The identifiers 154B may match the identifiers 154A. The identifiers 154B may be in an opposite order compared to the identifiers 154A. A portion of their body (e.g., arm, leg, limb, where the BFR device 100 is to be located, etc.) may be measured using the measuring tape 160 (e.g., the measuring tape 160 circumscribes the portion of the body) to identify the letter that the second distal end of the measuring tape 160 reaches. The strap 150 may be cut at the corresponding letter on the removable tape (e.g., after which the removable tape may be removed). The cut end of the strap 150 may be inserted into the alligator fit closure (e.g., portion 132B of closure system 130). The strap 150 may be sewn, have a hoop and/or loop material, and have a finished end (e.g., rounded, not cut).

FIGS. 12A-D illustrate cuffs 110 of the BFR devices 100, according to certain embodiments. FIGS. 12A-B illustrate a top view of a cuff 110 according to certain embodiments. The portion 132A of the closure system 130 may be pivotably attached (e.g., via a rivet that goes through at least a portion of the outer structure 114 and interior structure 112) to the cuff 110. The portion 132A of the closure system 130 may rotate about the pivot point. The rivet that secures the portion 132A to the cuff 110 may be coupled to one or more washers.

FIG. 12C illustrates a bottom view of a BFR device 100 that includes a cuff 110 and pad 140, according to certain embodiments. The cuff 110 may include one or more securing components 116 (e.g., elastic bands, elastic straps, etc.) configured to removably secure the pad 140 to the cuff 110. The pad 140 may have one or more corresponding recesses, each configured to receive a corresponding securing component 116.

FIG. 12D illustrates a side view of a BFR device 100 that includes a cuff 110, pad 140, and strap 150. The securing components 116 (e.g., elastic bands) of the cuff 110 may secure the pad 140 to the cuff 110. A first distal end (e.g., cut end) of a strap 150 may be secured by the portion 132B of the closure system 130 (e.g., alligator fit closure). A second distal end (e.g., rounded end) of the strap 150 may be routed through the portion 132A (e.g., D-ring) of the closure system 130 and may be secured to a portion of the strap 150 that was not routed through the portion 132A via a connection component 152 (e.g., brillo pad, a component that has a hook and/or loop material on the upper and lower surfaces). In some embodiments, the strap 150 may have excess length after being secured to itself via a connection component 152. For example, the same strap 150 may be used for different portions of a body that have different circumferences (e.g., arm and leg), so that when used on a small circumference, there is excess length. In another example, the same strap 150 may be used for different users (e.g., used in a clinic, fitness center, dwelling unit, etc. for different users) that have different circumferences (e.g., a first user has an arm of a first circumference and a second user has an arm of a second circumference that is different from the first circumference), so that when used on a smaller circumference, there is excess length. The excess length may be folded back and connected via a second connection component 152 to the strap 150. In some embodiments, the strap 150 may be cut to have a length so that a second connection component 152 is not used.

In some embodiments, different straps 150 of different lengths, materials, widths, thicknesses, etc. may be interchanged with the same cuff 110. A strap 150 may be attached to a cuff via one or more snaps, buttons, hook-and-loop materials, clips, and/or the like.

The alligator fit closure may open up into a “V” shape, where the interior of the “V” is a hook and/or loop material (e.g., Velcro® hook or loop) that interlocks with the hook and/or loop material on both sides of the strap 150 to secure the strap 150. The alligator fit closure may retract into the outer structure 114 of the cuff 110.

A D-ring (e.g., D-ring chafe) may allow for adjustments to positioning of the strap 150 that may eliminates need for a contoured strap.

The BFR device 100 may use a single rivet point with washers (e.g., below the interior structure 112 connected to the rivet that goes through the interior structure 112) coupled to the D-ring to allow the cuff 110 to make adjustments that provide a tighter fit to the contour of the upper portion of a limb.

The connection component 152 may secure the strap 150 without using a permanent material.

Signature pads 140 (e.g., designs, lettering, and/or the like on the face of the pad 140 that contacts the limb of the user) may be created by adding customer blanks to a pad mold 142.

A “Rate of Perceived Fatigue” may be used with the BFR device 100 to provide a more accurate measurement of effectiveness of blood-flow restriction training (BFRT) when applied to different use-cases. Tightening the BFR device 100 to generate pressure and an accompanying chart comparing the percentage of limb circumference may be used in combination with the inclusion of percentage of resistance and time to induce fatigue (e.g., which is the catalyst to deliver the benefits of BFRT).

The equation used may be percentage of arterial occlusion plus percentage of resistance (e.g., one repetition max (1RM)) plus duration (time) equals results. Conventional prediction of BFRT outcome may be limited to limb occlusion pressure (LOP). LOP is the pressure at which arterial flow is completely obstructed (e.g., typically 20 mm Hg above a person's measured systolic pressure). In practice, LOP can be determined using an acoustic Doppler on the distal side of the limb and noting when the heartbeat ceases to be detected. Surgeons may measure LOP at 100 mm HG over systolic for “bloodless” surgery.

Exercise in the form of resistance and repetitions has been shown to increase systolic between 30 mm Hg and 50 mm Hg which would increase the measured LOP. Conventionally, a large man weighing 250 pounds (lbs) with a systolic measurement of 120 may receive the same treatment as a 120 lb woman with a systolic reading of 112 even though give the same exercise, fatigue would be evident at different times using LOP as the only measurement. For example, put both on a treadmill setting incline to 5 degrees and speed to 5 miles per hour (mph) and the heavier man will fatigue first.

The activation of fatigue determines an outcome of either strength or endurance with BFRT and the release of growth hormone (GH), IGF1 (Insulin-Like Growth Factor 1), and the downregulation of myostatins, as well as many other body repair functions.

Measurement of fatigue is when the user cannot complete another repetition (rep). For example:

| | % Occlusion | % 1RM | Duration (SETS) |
|---|---|---|---|
| Post-Surgery | Low - 20% | Low - 20% | Long 5-10 min |
| Endurance | | | |
| Post-Surgery | Moderate - 40% | Moderate - 40% | Short - 60 seconds |
| Strength | | | |
| Hypertrophy | High - 80% | High - 80% | Short - 30 seconds |
| VO2 Max | Low - 30% | Low - 30% | Moderate - 15 min |
| Fitness | Moderate - 30% | Moderate - 30% | Moderate - 22 min |

The same table color coded is as follows:

| | % Occlusion | % 1RM | Duration |
|---|---|---|---|
| Post-Surgery | Green | Green | Red |
| Endurance | | | |
| Post-Surgery | Orange | Orange | Green |
| Strength | | | |
| Hypertrophy | Red | Red | Green |
| VO2 Max | Green | Green | Orange |
| Fitness | Orange | Orange | Orange |

The key for the tables may be as follows:

| Color Key | % Occlusion | % 1RM | Duration | |
|---|---|---|---|---|
| Green | 20% to 30% | 20% to 30% | >10 | minutes |
| Orange | 40% to 60% | 40% to 60% | <5 | minutes |
| Red | 65% to 85% | 65% to 85% | <1 | minute |

The BFR device 100 may be tightened based on one or more user characteristics (e.g., post-surgery endurance, post-surgery strength, hypertrophy, VO2 max, fitness, etc.). The amount of tightening (e.g., 20-30% occlusion, 40-60% occlusion, 65-85% occlusion) may be provided by rotating the bezel 126 to the corresponding tightness (e.g., see 20%, 40%, 60%, and 80% on tightening system 120 in FIG. 1C).

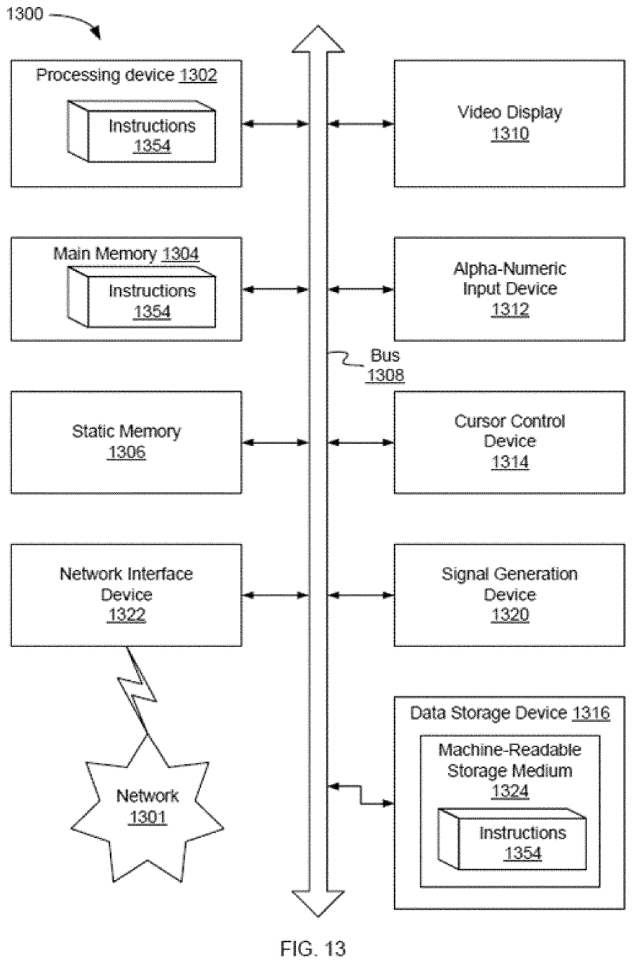

FIG. 13 illustrates a component diagram of a computer system which may implement one or more methods associated with a BFR device 100 described herein. A set of instructions for causing the computer system 1300 to perform any one or more of the methods discussed herein may be executed by the computer system 1300. In some embodiments, the computer system 1300 may implement the functions of the microcontroller 320 of FIG. 3.

In one embodiment, the computer system 1300 may be connected to other computer systems by a network 1301 provided by a Local Area Network (LAN), an intranet, an extranet, the Internet or any combination thereof. The computer system may operate in the capacity of a server or a client machine in a client-server network environment or as a peer machine in a peer-to-peer (or distributed) network environment. The computer system may be a personal computer (PC), a tablet PC, a set-top box (STB), a Personal Digital Assistant (PDA), a cellular telephone, a web appliance, a server, a network router, switch, bridge or any machine capable of executing a set of instructions (sequential or otherwise) that specify actions to be taken by that machine. Further, while a single machine is illustrated, the term “computer system” shall also be taken to include any collection of machines (e.g., computers) that individually or jointly execute a set (or multiple sets) of instructions to perform any one or more of the methodologies discussed herein.

In one embodiment, the computer system 1300 includes a processing device 1302 (e.g., microcontroller), a main memory 1304 (e.g., read-only memory (ROM), flash memory, dynamic random access memory (DRAM) such as synchronous DRAM (SDRAM), etc.), a static memory 1306 (e.g., flash memory, static random access memory (SRAM), etc.) and a data storage device 1316, which communicate with each other via a bus 1308.

In one embodiment, the processing device 1302 represents one or more general-purpose processors such as a microprocessor, central processing unit or the like. Processing device may include any combination of one or more integrated circuits and/or packages that may, in turn, include one or more processors (e.g., one or more processor cores). Therefore, the term processing device encompasses a single core CPU, a multi-core CPU and a massively multi-core system that includes many interconnected integrated circuits, each of which may include multiple processor cores. The processing device 1302 may therefore include multiple processors. The processing device 1302 may include a complex instruction set computing (CISC) microprocessor, reduced instruction set computing (RISC) microprocessor, very long instruction word (VLIW) microprocessor, processor implementing other instruction sets or processors implementing a combination of instruction sets. The processing device 1302 may also be one or more special-purpose processing devices such as an application specific integrated circuit (ASIC), a field programmable gate array (FPGA), a digital signal processor (DSP), network processor or the like.

The processing device 1302 may be the microcontroller 320 of FIG. 3. The processing device 1302 may perform one or more methods associated with the BFR device 100 (e.g., receiving sensor date from the sensor 310, processing the sensor data, transmitting the sensor data, causing the sensor data to be displayed, providing an alert based on the sensor data, and/or the like). The processing device 1302 may include one or more interfaces to connect to the sensor 310.

In one embodiment, the computer system 1300 may further include one or more network interface devices 1322. The computer system 1300 also may include a video display unit 1310 (e.g., a liquid crystal display (LCD) or a cathode ray tube (CRT)), an alphanumeric input device 1312 (e.g., a keyboard), a cursor control device 1314 (e.g., a mouse) and a signal generation device 1320 (e.g., a speaker).

In one embodiment, the data storage device 1318 may include a computer-readable storage medium 1324 on which is stored one or more sets of instructions 1354 embodying any one or more of the methods or functions described herein. The instructions 1354 may also reside, completely or at least partially, within the main memory 1304 and/or within the processing device 1302 during execution thereof by the computer system 1300; the main memory 1304 and the processing device 1302 also constituting machine-readable storage media. The computer-readable storage medium 1324 may be a non-transitory computer-readable storage medium.

While the computer-readable storage medium 1324 is shown as a single medium, the term “computer-readable storage medium” should be taken to include a single medium or multiple media (e.g., a centralized or distributed database and associated caches and servers) that store the one or more sets of instructions. The term “computer-readable storage medium” shall also be taken to include any medium that is capable of storing, encoding, or carrying a set of instructions for execution by the machine and that cause the machine to perform any one or more of the methods described herein. Examples of computer-readable storage media include, but not limited to, solid-state memories, optical media and magnetic media. The preceding description sets forth numerous specific details such as examples of specific system, components, devices and so forth in order to provide a good understanding of several embodiments of the present disclosure. It will be apparent to one skilled in the art, however, that at least some embodiment of the present disclosure may be practiced without these specific details. In other instances, well-known components or methods are not described in detail to avoid unnecessarily obscuring the present disclosure. Thus, the specific details set forth are merely exemplary. Particular implementations may vary from these exemplary details and still be contemplated to be within the scope of the present disclosure.

It should be borne in mind, however, that all of these and similar terms are to be associated with the appropriate physical quantities and are merely convenient labels applied to these quantities. Unless specifically stated otherwise as apparent from the above discussion, it is appreciated that throughout the description, discussions utilizing terms such as “receiving,” “transmitting,” “causing,” “providing,” “displaying,” or the like, refer to the actions and processes of a computer system, or similar electronic computing device, that manipulates and transforms data represented as physical (e.g., electronic) quantities within the computer system's registers and memories into other data similarly represented as physical quantities within the computer system memories or registers or other such information storage, transmission or display devices.

Embodiments also relate to an apparatus for performing the operations herein. This apparatus may be specially constructed for the required purposes, or it may include a general-purpose computer selectively activated or reconfigured by a computer program stored in the computer. Such a computer program may be stored in a computer readable storage medium, such as, but not limited to, any type of disk including floppy disks, optical disks, CD-ROMs and magnetic-optical disks, read-only memories (ROMs), random access memories (RAMs), EPROMs, EEPROMs, magnetic or optical cards, or any type of media suitable for storing electronic instructions.

The algorithms and displays presented herein are not inherently related to any particular computer or other apparatus. Various general-purpose systems may be used with programs in accordance with the teachings herein, or it may prove convenient to construct a more specialized apparatus to perform the required method steps. The required structure for a variety of these systems will appear from the description below. In addition, the present embodiments are not described with reference to any particular programming language. It will be appreciated that a variety of programming languages may be used to implement the teachings of the present invention as described herein. It should also be noted that the terms “when” or the phrase “in response to,” as used herein, should be understood to indicate that there may be intervening time, intervening events, or both before the identified operation is performed.

The above description of illustrated embodiments of the disclosure, including what is described in the Abstract, is not intended to be exhaustive or to limit the disclosure to the precise forms disclosed. While specific embodiments of, and examples for, the disclosure are described herein for illustrative purposes, various equivalent modifications are possible within the scope of the disclosure, as those skilled in the relevant art will recognize.

Various operations are described as multiple discrete operations, in turn, in a manner that is most helpful in understanding the present disclosure, however, the order of description should not be construed to imply that these operations are necessarily order dependent. In particular, these operations need not be performed in the order of presentation.

The terms “over,” “under,” “between,” “disposed on,” and “on” as used herein refer to a relative position of one material layer or component with respect to other layers or components. For example, one layer disposed on, over, or under another layer may be directly in contact with the other layer or may have one or more intervening layers. Moreover, one layer disposed between two layers may be directly in contact with the two layers or may have one or more intervening layers. Similarly, unless explicitly stated otherwise, one feature disposed between two features may be in direct contact with the adjacent features or may have one or more intervening layers. Some features may be incorporated into intervening layers.

While the present disclosure has been described with respect to a limited number of embodiments, those skilled in the art will appreciate numerous modifications and variations therefrom. It is intended that the appended claims cover all such modifications and variations as fall within the true spirit and scope of this present disclosure.

In the description herein, numerous specific details are set forth, such as examples of specific measurements/heights in order to provide a thorough understanding of the present disclosure. It will be apparent, however, to one skilled in the art that these specific details need not be employed to practice the present disclosure. In other instances, well known components or methods, such as specific manufacturing techniques and materials have not been described in detail in order to avoid unnecessarily obscuring the present disclosure.

Use of the phrase ‘configured to,’ in one embodiment, refers to arranging, putting together, manufacturing, offering to sell, importing and/or designing an apparatus, hardware, logic, or element to perform a designated or determined task. In this example, an apparatus or element thereof that is not operating is still ‘configured to’ perform a designated task if it is designed, coupled, and/or interconnected to perform said designated task. As a purely illustrative example, a logic gate can provide a 0 or a 1 during operation. But a logic gate ‘configured to’ provide an enable signal to a clock does not include every potential logic gate that can provide a 1 or 0. Instead, the logic gate is one coupled in some manner that during operation the 1 or 0 output is to enable the clock. Note once again that use of the term ‘configured to’ does not require operation, but instead focus on the latent state of an apparatus, hardware, and/or element, where in the latent state the apparatus, hardware, and/or element is designed to perform a particular task when the apparatus, hardware, and/or element is operating.

Furthermore, use of the phrases ‘to,’ ‘capable of/to,’ and or ‘operable to,’ in one embodiment, refers to some apparatus, logic, hardware, and/or element designed in such a way to enable use of the apparatus, logic, hardware, and/or element in a specified manner. Note as above that use of to, capable to, or operable to, in one embodiment, refers to the latent state of an apparatus, logic, hardware, and/or element, where the apparatus, logic, hardware, and/or element is not operating but is designed in such a manner to enable use of an apparatus in a specified manner.

Reference throughout this specification to “some embodiments,” “one embodiment,” or “an embodiment” means that a particular feature, structure, or characteristic described in connection with the embodiment is included in at least one embodiment of the present disclosure. Thus, the appearances of the phrases “in some embodiments,” “in one embodiment,” or “in an embodiment” in various places throughout this specification are not necessarily all referring to the same embodiment. Furthermore, the particular features, structures, or characteristics can be combined in any suitable manner in one or more embodiments and that the use of any specified element, component, or device is not isolated to the exemplary embodiment within where it is described. In addition, the term “or” is intended to mean an inclusive “or” rather than an exclusive “or.” When the term “about”, “approximately”, or “substantially” is used herein, this is intended to mean the nominal value or characteristic presented is precise within ±10%.

In the foregoing specification, a detailed description has been given with reference to specific exemplary embodiments. It will, however, be evident that various modifications and changes can be made thereto without departing from the broader spirit and scope of the disclosure as set forth in the appended claims. The specification and drawings are, accordingly, to be regarded in an illustrative sense rather than a restrictive sense. Furthermore, the foregoing use of embodiment and other exemplarily language does not necessarily refer to the same embodiment or the same example, but can refer to different and distinct embodiments, as well as potentially the same embodiment.

The words “example” or “exemplary” are used herein to mean serving as an example, instance or illustration. Any aspect or design described herein as “example” or “exemplary” is not necessarily to be construed as preferred or advantageous over other aspects or designs. Rather, use of the words “example” or “exemplary” is intended to present concepts in a concrete fashion. As used in this application, the term “or” is intended to mean an inclusive “or” rather than an exclusive “or.” That is, unless specified otherwise, or clear from context, “X includes A or B” is intended to mean any of the natural inclusive permutations. That is, if X includes A; X includes B; or X includes both A and B, then “X includes A or B” is satisfied under any of the foregoing instances. In addition, the articles “a” and “an” as used in this application and the appended claims should generally be construed to mean “one or more” unless specified otherwise or clear from context to be directed to a singular form. Moreover, use of the term “an embodiment” or “one embodiment” or “an implementation” or “one implementation” throughout is not intended to mean the same embodiment or implementation unless described as such. Also, the terms “first,” “second,” “third,” “fourth,” etc. as used herein are meant as labels to distinguish among different elements and can not necessarily have an ordinal meaning according to their numerical designation.

What is claimed is:

1. A blood flow restriction (BFR) device for circumscribing a portion of a body, the BFR device comprising:
a cuff;
a tightening system coupled to the cuff, wherein the tightening system is configured to tighten and loosen the cuff; and
a silicon-based cushion disposed on an inner surface of the cuff.

2. The BFR device of claim 1, wherein the cuff comprises a first distal cuff end, a second distal cuff end, and a closure system to couple the first distal cuff end to the second distal cuff end for circumscribing the portion of the body, the closure system comprising one or more of a quick-fit strap closure, an alligator fit closure, or a D-ring.

3. The BFR device of claim 1, wherein the silicon-based cushion is configured to partially occlude arterial flow while occluding venous flow.

4. The BFR device of claim 1, wherein the silicon-based cushion is a platinum-based silicon material.

5. The BFR device of claim 1 further comprising:
a strap comprising:
a first portion configured to removably attach to a first distal cuff end of the cuff; and
a second portion configured to removably attach to a second distal cuff end of the cuff.

6. The BFR device of claim 5, wherein:
the first distal cuff end comprises an alligator fit closure comprising an upper alligator portion and a lower alligator portion; and
to attach the first portion to the alligator fit closure, a lower strap surface of the first portion of the strap is to be disposed on an upper surface of the lower alligator portion and a lower surface of the upper alligator portion is to be disposed on an upper strap surface the first portion of the strap.

7. The BFR device of claim 6, wherein the second distal cuff end comprises a D-ring rotatably attached to the cuff, and wherein the second portion of the strap is configured to be routed through the D-ring and removably attached to a third portion of the strap via a connection component.

8. The BFR device of claim 7, wherein:
the upper strap surface comprises a first type of material configured to secure to a second type of material;
the lower strap surface comprises the first type of material;
the upper surface of the lower alligator portion comprises the second type of material;
the lower surface of the upper alligator portion comprises the second type of material; and
the connection component comprises the second type of material.

9. The BFR device of claim 1, wherein the silicon-based cushion is coupled to the inner surface of the cuff via elastic straps of the cuff

10. The BFR device of claim 1, wherein the silicon-based cushion forms ridges and recesses configured to compress together responsive to tightening of the BFR device.

11. A blood flow restriction (BFR) device to circumscribe a portion of a body, the BFR device comprising:
a cuff comprising an outer structure and an interior structure; and
a tightening system comprising:
a pull wire disposed between the outer structure and the interior structure;
a spool coupled to the pull wire; and
a bezel disposed on the spool, wherein the bezel is configured to rotate to tighten the BFR device.

12. The BFR device of claim 11, wherein the cuff comprises a first distal cuff end, a second distal cuff end, and a closure system to couple the first distal cuff end to the second distal cuff end for circumscribing the portion of the body, the closure system comprising one or more of a quick-fit strap closure, an alligator fit closure, or a D-ring.

13. The BFR device of claim 11, wherein the BFR device is configured to partially occlude arterial flow while occluding venous flow.

14. The BFR device of claim 11, wherein:
the tightening system further comprises a base attached to the interior structure;
the spool is disposed on the base;
at least a portion of the tightening system extends through an opening in the outer structure;
the bezel is disposed above the interior structure and the outer structure.

15. The BFR device of claim 11, wherein:
the BFR device further comprises a rivet attached to the interior structure; and
a first distal end of the pull wire is coupled to the rivet and a second distal end of the pull wire is coupled to the spool.

16. The BFR device of claim 11 further comprising:
a strap comprising:
a first portion configured to removably attach to a first distal cuff end of the cuff; and
a second portion configured to removably attach to a second distal cuff end of the cuff; and
a hanger coupled to the first portion of the strap, wherein the pull wire is routed through the hanger, wherein the first portion of the strap is pulled into the cuff responsive to rotation of the bezel.

17. A blood flow restriction (BFR) device for circumscribing a portion of a body, the BFR device comprising:
a cuff;
a tightening system coupled to the cuff, wherein the tightening system is configured to tighten and loosen of the cuff; and
a limb occlusion pressure (LOP) sensor disposed on an inner surface of the cuff, wherein the LOP sensor is to provide an indication of one or more of an LOP reading, active pressure, or pulse.

18. The BFR device of claim 17, wherein the cuff comprises a first distal cuff end, a second distal cuff end, and a closure system to couple the first distal cuff end to the second distal cuff end for circumscribing the portion of the body, the closure system comprising one or more of a quick-fit strap closure, an alligator fit closure, or a D-ring.

19. The BFR device of claim 17, wherein the BFR device is configured to partially occlude arterial flow while occluding venous flow.

20. The BFR device of claim 17 further comprising a microcontroller coupled to the LOP sensor, wherein the microcontroller is to receive the LOP reading and cause the LOP reading to be displayed.
